(19) United States
(12) Patent Application Publication
(10) Pub. No.: US 2015/0366884 A1
(43) Pub. Date: Dec. 24, 2015

(54) COMPOSITIONS AND METHODS FOR CANCER THERAPY

(71) Applicants: Michael K. SCHULTZ, Iowa City, IA (US); Kyle C. KLOEPPING, Iowa City, IA (US); Douglas R. SPITZ, Iowa City, IA (US); UNIVERSITY OF IOWA RESEARCH FOUNDATION, Iowa City, IA (US)

(72) Inventors: Michael K. SCHULTZ, Iowa City, IA (US); Kyle C. KLOEPPING, Iowa City, IA (US); Douglas R. SPITZ, Iowa City, IA (US)

(73) Assignee: UNIVERSITY OF IOWA RESEARCH FOUNDATION, Iowa City, IA (US)

(21) Appl. No.: 14/766,699

(22) PCT Filed: Feb. 10, 2014
(86) PCT No.: PCT/US14/15611

§ 371 Date: Aug. 7, 2015

Related U.S. Application Data
(60) Provisional application No. 61/762,771, filed on Feb. 8, 2013.

(51) Int. Cl. A61K 31/675; A61K 31/375; A61K 31/198; A61K 31/7135; A61K 45/06; A61K 31/66

(52) U.S. Cl. CPC A61K 31/675; A61K 45/06; A61K 31/66; A61K 31/198; A61K 31/7135; A61K 31/375

(57) ABSTRACT

The invention provides compositions and methods to treat cancer with an agent that selectively promotes cancer cell death relative to non-malignant cells by mechanisms that include increased oxidative stress (“a therapeutic agent”) or a pharmaceutically acceptable salt thereof, an inhibitor of hydroperoxide metabolism and a pharmaceutically acceptable diluent or carrier.

### RELATED APPLICATION

This application claims priority to U.S. Provisional Patent Application No. 61/762,771, filed Feb. 8, 2013, the entirety of which is incorporated herein by reference.

### FEDERAL GRANT SUPPORT

The invention was made with Government support under US NRC-HQ-12-G-38-0041 awarded by the US Nuclear Regulatory Commission. The government has certain rights in the invention.

### BACKGROUND

Most treatment plans for patients with cancer include surgery, radiation therapy, and/or chemotherapy. However, because of problems with such treatment plans, such as side-effects caused by radiation therapy and chemotherapy, additional methods are needed for treating cancer.

### SUMMARY

The present invention provides a pharmaceutical composition comprising a cancer cell specific cytotoxicity agent that targets cancer cell mitochondria and preferentially kills cancer cells by mechanisms that may include increased reactive oxygen species (ROS) levels in cancer cells relative to non-malignant cells, and a pharmaceutically acceptable diluent or carrier. The composition of matter may also inhibit/promote specific cancer cell pathways that induce cancer cell specific cell death relative to non-malignant cells. Examples of reactive oxygen species include superoxide and hydrogen peroxide (i.e., O₂^{•-}, H₂O₂). The present compounds have cancer cell specific cytotoxicity without the addition of a hydroperoxide species metabolism inhibitor.

In certain embodiments, the present invention provides a pharmaceutical composition comprising a compound of formula I:

Ph₃P⁺-L-W Y⁻ I

wherein:

W is selected from:

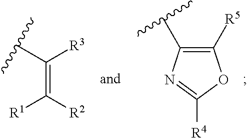

L is absent, (C₁-C₁₂)alkyl, (C₁-C₁₂)alkylene, —(CH₂CH₂O)ₙM-, —C(═O)NR^{L1}— or —NR^{L1}C(═S)NR^{L1}—

n is 1 to 12;

M is absent or —CH₂CH₂—;

R^{L1} is H or (C₁-C₆)alkyl;

R¹ is halo or —NHC(═O)Rₐ;

R² is halo, SR_{b} or —C(═O)NHR_{c};

R³ is —NH(C═O)R_{d}, —NH(C═O)NHR_{d} or phenyl wherein any phenyl of R³ is optionally substituted with one or more halo, (C₁-C₃)alkyl, (C₁-C₃)haloalkyl or —O(C₁-C₃)alkyl;

R⁴ is (C₁-C₆)alkyl or phenyl wherein any phenyl of R⁴ is optionally substituted with one or more halo, (C₁-C₃)alkyl, (C₁-C₃)haloalkyl or —O(C₁-C₃)alkyl;

R⁵ is —S(C₁-C₆)alkyl or —N((C₁-C₆)alkyl)₂;

Rₐ is phenyl optionally substituted with one or more halo, (C₁-C₃)alkyl, (C₁-C₃)haloalkyl or —O(C₁-C₃)alkyl;

R_{b} is phenyl optionally substituted with one or more halo, (C₁-C₃)alkyl, (C₁-C₃)haloalkyl or —O(C₁-C₃)alkyl;

R_{c} is phenyl optionally substituted with one or more halo, (C₁-C₃)alkyl, (C₁-C₃)haloalkyl or —O(C₁-C₃)alkyl;

R_{d} is phenyl optionally substituted with one or more halo, (C₁-C₃)alkyl, (C₁-C₃)haloalkyl or —O(C₁-C₃)alkyl; and

Y is a counterion;

or a pharmaceutically acceptable salt thereof, and a pharmaceutically acceptable diluent or carrier.

In certain embodiments, the present invention provides a pharmaceutical composition comprising a compound of formula I:

Ph₃P⁺-L-W Y⁻ I

wherein:

W is selected from:

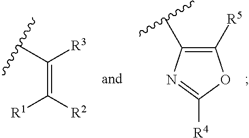

L is absent, (C₁-C₂₀)alkyl, (C₁-C₂₀)alkylene, —(CH₂CH₂O)ₙM-, —C(═O)NR^{L1}— or —NR^{L1}C(═S)NR^{L1}—

n is 1 to 20;

M is absent or —CH₂CH₂—;

R^{L1} is H or (C₁-C₆)alkyl;

R¹ is halo or —NHC(═O)Rₐ;

R² is halo, SR_{b} or —C(═O)NHR_{c};

R³ is —NH(C═O)R_{d}, —NH(C═O)NHR_{d} or phenyl wherein any phenyl of R³ is optionally substituted with one or more halo, (C₁-C₃)alkyl, (C₁-C₃)haloalkyl or —O(C₁-C₃)alkyl;

R⁴ is (C₁-C₆)alkyl or phenyl wherein any phenyl of R⁴ is optionally substituted with one or more halo, (C₁-C₃)alkyl, (C₁-C₃)haloalkyl or —O(C₁-C₃)alkyl;

R⁵ is —S(C₁-C₆)alkyl or —N((C₁-C₆)alkyl)₂;

Rₐ is phenyl optionally substituted with one or more halo, (C₁-C₃)alkyl, (C₁-C₃)haloalkyl or —O(C₁-C₃)alkyl;

R_{b} is phenyl optionally substituted with one or more halo, (C₁-C₃)alkyl, (C₁-C₃)haloalkyl or —O(C₁-C₃)alkyl;

R_{c} is phenyl optionally substituted with one or more halo, (C₁-C₃)alkyl, (C₁-C₃)haloalkyl or —O(C₁-C₃)alkyl;

R_{d} is phenyl optionally substituted with one or more halo, (C₁-C₃)alkyl, (C₁-C₃)haloalkyl or —O(C₁-C₃)alkyl; and

Y is a counterion;

or a pharmaceutically acceptable salt thereof, and a pharmaceutically acceptable diluent or carrier.

In certain embodiments, the compound of formula I is hexadecyl-TPP.

As used herein, the term triphenylphosphonium (TPP) is any molecule containing a triphenylphosphine cation (⁺PPh₃) moiety.

As used herein halo is fluoro, chloro, bromo, or iodo.

As used herein, the term alkyl is defined as a straight or branched hydrocarbon. For example, an alkyl group can have 1 to 12 carbon atoms (i.e, (C₁-C₁₂)alkyl), 1 to 10 carbon atoms (i.e., (C₁-C₁₀)alkyl), 1 to 8 carbon atoms (i.e., (C₁-C₈)alkyl) or 1 to 6 carbon atoms (i.e., (C₁-C₆ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂) and decyl (—(CH₂)₉CH₃). As used herein, the term alkylene is an unsubstituted alkyl. As used herein, the term haloalkyl is an alkyl substituted with a halo. As used herein the term counterion is a pharmaceutically acceptable counterion such as a pharmaceutically acceptable anion (e.g. Cl⁻, Br⁻, I⁻, CH₃SO₃⁻, CF₃SO₃⁻ or p-CH₃C₆H₄ SO₃).

In certain embodiments, the present invention provides a pharmaceutical composition comprising a therapeutic agent that targets cancer cell mitochondria and interrupting electron transport activity, thereby increasing reactive oxygen species (ROS) levels in cancer cell mitochondria and providing an intervention that promotes cancer cell specific cell death by disrupting other cancer cell survival pathways, wherein the therapeutic agent is a compound of Formula I or a pharmaceutically acceptable salt thereof, and a pharmaceutically acceptable diluent or carrier. In certain embodiments, the pharmaceutical composition further comprises an inhibitor of glutathione synthesis or hydroperoxide metabolism comprising L-buthionine-[S,R]-sulfoximine (BSO), (S-triethylphosphinegold(I)-2,3,4,6-tetra-O-acetyl-1-thio-β-D-glucopyranoside Auranofin (AUR), or a combination of BSO and AUR. Other compounds that could also be used for this purpose include inhibitors of catalase (i.e. 3-aminotriazole), inhibitors of glucose metabolism (i.e., bromopyruvate and 2-deoxyglucose), inhibitors of peroxiredoxins, inhibitors of glutathione peroxidases, inhibitors of dehydrogenase enzymes that regenerate NADPH, inhibitors of thioredoxin reductase, inhibitors of glutathione reductase, inhibitors of glutathione transferases, and inhibitors of transcription factors as well as signal transduction proteins that regulate thiol mediated hydroperoxide metabolism (i.e., Nrf-2, AP-1, NFkB, AKT, ERK1/2, p38, EGFR, and IGFR). Another strategy to enhance the efficacy of a composition including DTPP with inhibitors of hydroperoxide metabolism would include feeding patients diets high in respiratory directed substrates including ketogenic diets, Atkins style diets, and pharmacological doses of IV vitamin C which would be expected to further enhance the differential metabolic production of pro-oxidants in cancer vs. normal tissues.

The present invention provides a method for treating cancer in a mammal, comprising administering a composition comprising the compound of Formula I to the mammal. In certain embodiments, the therapeutic agent increases reactive oxygen species (ROS) levels in cancer cell mitochondria. In certain embodiments, the therapeutic agent is administered in conjunction with an inhibitor of hydroperoxide metabolism or glutathione synthesis, either sequentially or in a single composition.

The present invention provides a method for inducing cancer cell specific clonogenic cell killing and cellular apoptosis of a cancerous cell, comprising contacting the cancerous cell with an effective clonogenic cell killing or apoptosis-inducing amount of the composition comprising the compound of Formula I. In certain embodiments, a therapeutic agent is administered in conjunction with an inhibitor of hydroperoxide metabolism or glutathione synthesis, either sequentially or in a single composition or with another cancer therapy to enhance the effect of that cancer therapy or to induce cancer cell specific cell death synergistically.

The present invention provides a method for increasing the anticancer effects of a conventional cancer therapy (i.e., radio- and/or chemo-therapy) on cancerous cells in a mammal, comprising contacting the cancerous cell with an effective amount of the composition comprising the compound of Formula I and administering an additional conventional cancer therapy modality. In certain embodiments, the additional cancer therapy is chemotherapy and/or radiation. In certain embodiments, a therapeutic agent is administered in conjunction with an inhibitor of hydroperoxide metabolism or glutathione synthesis, either sequentially or in a single composition.

In certain embodiments of the methods described above, the composition does not significantly inhibit viability of comparable non-cancerous cells.

The present invention provides a method for selectively inducing cancer cell specific cell death relative to non-malignant cells by mechanisms including increased oxidative stress, in a cancer cell in a mammal in need of such treatment comprising administering to the mammal an effective amount of the composition described above. In certain embodiments, a therapeutic agent is administered in conjunction with an inhibitor of hydroperoxide metabolism or glutathione synthesis, either sequentially or in a single composition. In certain embodiments, the mammal is a human.

In certain embodiments of the methods described above, the cancer is breast cancer, prostate cancer, lung cancer, pancreas cancer, head and neck cancer, ovarian cancer, brain cancer, colon cancer, hepatic cancer, skin cancer, leukemia, melanoma, endometrial cancer, neuroendocrine tumors, carcinoids, neuroblastoma, tumors arising from the neural crest, lymphoma, myeloma, or other malignancies characterized by aberrant mitochondrial hydroperoxide metabolism. In certain embodiments, the cancer is the above cancers that are not curable or not responsive to other therapies. In certain embodiments the cancers are hormone dependent or hormone-independent epithelial cancers.

In certain embodiments of the methods described above, the tumor is reduced in volume by at least 10%. In certain embodiments, the tumor is reduced by any amount between 1-100%. In certain embodiments, the tumor uptake of molecular imaging agents, such as fluorine-18 deoxyglucose, fluorine-18 thymidine or other suitable molecular imaging agent, is reduced by any amount between 1-100%. In certain embodiments the imaging agent is fluorine-18 deoxyglucose, fluorine-18 thymidine or other suitable molecular imaging agent. In certain embodiments, the mammal's symptoms (such as flushing, nausea, fever, or other maladies associated with cancerous disease) are alleviated.

In certain embodiments of the methods described above, the composition is administered intraveneously, orally, subcutaneously, interperitoneally, or as an aerosol. In certain embodiments of the methods described above, the composition is administered intraveneously at a dosage of 5-200 micromols/kg/day of therapeutic agent, such as 20-130 micromols/kg/day of therapeutic agent. In certain embodiments of the methods described above, the composition is administered orally at a dosage of 5-200 micromols/kg/day of therapeutic agent, such as 20-130 micromols/kg/day of therapeutic agent.

The present invention provides a method for treating cancer in a subject, comprising administering to the subject an effective amount of therapeutic agent and an inhibitor or inhibitors of hydroperoxide metabolism and/or an inhibitor of glutathione metabolism so as to treat the cancer.

In certain embodiments, the present invention provides a composition comprising a compound of Formula I or a pharmaceutically acceptable salt thereof, and an inhibitor of hydroperoxide metabolism for use in the treatment of cancer, wherein the composition is to be administered to a patient that has cancer or is at risk for developing cancer.

In certain embodiments, the present invention provides a composition comprising a compound of Formula I or a pharmaceutically acceptable salt thereof, and an inhibitor of hydroperoxide metabolism for use in inducing cellular apoptosis of a cancerous cell, wherein the composition is to be administered to a patient that has cancer or is at risk for developing cancer.

### BRIEF DESCRIPTION OF THE DRAWINGS AND TABLES

FIG. 1: One Proposed Mechanism of TPP-drug induced cancer cell specific cell killing via oxidative stress: (A) The positively charged TPP head preferentially translocates TPP to the mitochondrial membrane due to the hyperpolarized membrane gradient in cancer cell mitochondria; (B) The side chain (a 10 carbon aliphatic 10-TPP shown here) embeds into the inner mitochondrial membrane, disrupting ETC complexes (drawn here at complex III) leading to increased one electron reductions of O₂ to form superoxide (O₂^{•-}) and hydrogen peroxide (H₂O₂); (C) H₂O₂ is metabolized by cellular scavengers [e.g., glutathione peroxidases (Gpx) and peroxiredoxins (Prx)]; (D) one proposed combination therapy includes inhibitors of H₂O₂ metabolism (BSO and AUR) leading to cancer cell death via increased oxidative stress.

FIG. 2: TPP-based compounds that selectively kill melanoma cells relative to non-malignant melanocytes. Ten TPP-based compounds identified by HTS that show selective cytotoxicity toward cancer cells relative to non-maligant cells (melanocytes). Compounds grouped by TPP-sidechain functional-group-similarity display similar cytotoxicity profiles. Normalized surviving fraction (NSF) of live cells were determined by the MTT assay (24 hour incubation; 2 μM). Melanoma cells were BRAF mutant A375 (n=2). Dark bars are for non-malignant melanocytes (n=1). Uncertainties shown are ±SEM. These data show that HTS can be used to identify TPP based drugs that selectively kill melanoma cells relative to normal cells.

FIG. 3: TPP Based Compounds of Formula I that display cancer cell specific cytotoxicity relative to non-malignant cells.

FIG. 4. Screen of 46 TPP based compounds to determine the relative cytotoxicity of the compounds to cancer cells relative to non-malignant cells. MTT survival assays were performed on cells that were incubated for 24 hours at a concentration of 2 μM with TPP-based compounds. Gray bars represent the average of duplicate measurements of non-malignant normal melanocytes treated with the TPP-based compound library. Black bars represent the surviving fraction of malignant BRAF mutant A375 melanoma cells under the same conditions. These data demonstrate the specific structural characteristics of TPP-based compound side chains can be identified that impart cancer cell specific cytotoxicity relative to non-malignant cells.

FIGS. 5A-5C. MTT viability analysis of melanoma cells versus non-malignant melanocyte cells looking at the effect of variation in TPP molecular chain length and treatment time. A375 human melanoma cells in (A), MeWo human melanoma cells in (B), and non-malignant melanocyte cells in (C).

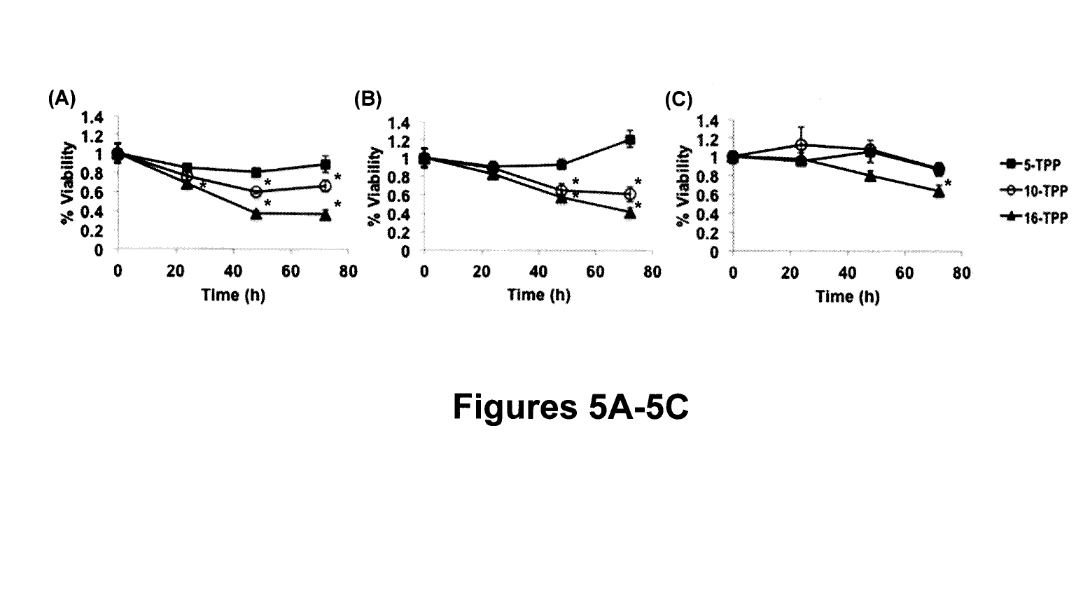

### DETAILED DESCRIPTION

Despite treatment advances that have reduced overall cancer mortality, deaths from melanoma increased 619% from 1950 to 2004. A major reason for this is that no systemic therapy provides durable benefit to metastatic melanoma patients. Systemic therapies induce tumor response in only 5-20% of patients and combination therapies have not improved survival significantly. Recent immune system enhancement and protein kinase pathway treatments are encouraging, but durable benefit remains elusive. The mechanism of resistance is debated, but appears to comprise innate melanoma cell ability to avoid immune surveillance; recognize and export drugs; and may involve “stem-like” cells that are particularly drug resistant. In 2011, two new therapies were FDA approved, but extend life expectancy by only months in a fraction of patients that possess specific gene mutations.

The present drug development is directed toward developing a new class of drugs that exploit differences in oxidative metabolism and susceptibility to mitochondrial intervention of melanoma cells relative to non-malignant cells to selectively kill melanoma cells. This contribution is significant because it exploits differences in oxidative metabolism to circumvent melanoma multi-drug resistance and immune response avoidance to provide durable benefit for a rapidly increasing number of metastatic melanoma patients for whom current therapies fall short. Further, this research is significant because patients diagnosed with melanoma are relatively young and raising children (median age is 45 at diagnosis). Finally, because peculiarities in cancer cell mitochondrial oxidative metabolism and susceptibility to mitochondrial targeted interventions are emerging as general cancer therapy targets, the research has the potential to be broadly applied.

Emerging evidence suggests fundamental differences in mitochondrial oxidative metabolism of cancer cells relative to normal cells leads to increased steady-state levels of reactive oxygen species (ROS) such as hydroperoxides. Furthermore, upregulated glucose metabolism in cancer cells relative to normal cells (including melanoma) is increasingly recognized as a mechanism that not only supports increased energy needs, but also detoxifies hydroperoxides in cancer cells via GSH-dependent peroxidases and deacetylation reactions involving pyruvate. Another consequence of disrupted mitochondrial metabolism in cancer cells is hyperpolarization of cancer cell mitochondrial membranes, relative to normal cells.

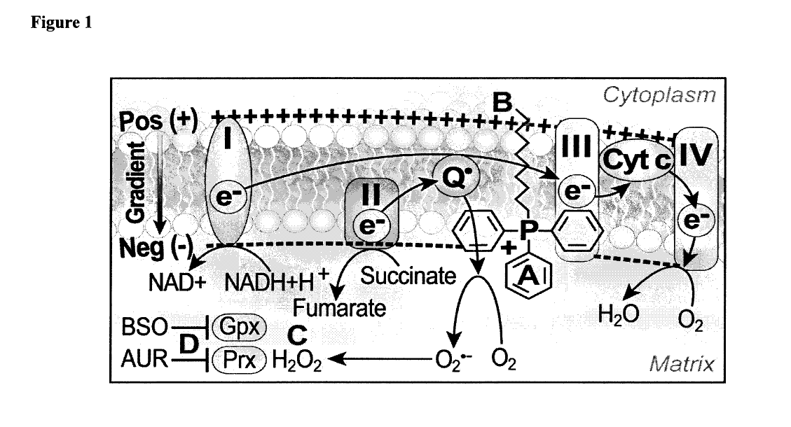

The drug delivery vehicle used in the present work is a triphenylphosphonium (TPP) moiety that preferentially translocates into tumor cell mitochondria (via attraction to the hyperpolarized membrane potential) by way of a delocalized positive charge at the lipophilic triphenyl-targeting “head” (FIG. 1). (Fath et al., Mitochondrial electron transport chain blockers enhance 2-deoxy-D-glucose induced oxidative stress and cell killing in human colon carcinoma cells. Cancer Biol Ther, 2009. 8(13):1228-1236. PMID:19411865; Ross et al., Lipophilic triphenylphosphonium cations as tools in mitochondrial bioenergetics and free radical biology. Biochemistry (Mosc), 2005. 70(2):222-230. PMID:15807662; Ross et al., Rapid and extensive uptake and activation of hydrophobic triphenylphosphonium cations within cells. Biochem J, 2008. 411(3):633-645. PMID:18294140; Ojovan et al., Accumulation of dodecyltriphenylphosphonium in mitochondria induces their swelling and ROS-dependent growth inhibition in yeast. J Bioenerg Biomembr, 2011. 43(2):175-180. PMID:21360288.) In addition, fundamental defects in cancer cell mitochondrial oxidative metabolism are increasingly recognized as rendering them more vulnerable to agents that induce oxidative stress. (Aykin-Burns et al., Increased levels of superoxide and H₂O₂ mediate the differential susceptibility of cancer cells versus normal cells to glucose deprivation. Biochem J, 2009. 418(1):29-37. PMID:18937644; Pelicano et al., Inhibition of mitochondrial respiration: a novel strategy to enhance drug-induced apoptosis in human leukemia cells by a reactive oxygen species-mediated mechanism. J Biol Chem, 2003. 278(39):37832-37839. PMID:12853461.) The data show that TPP-based drugs can be designed to increase mitochondrial superoxide (O₂^{•-}) production in melanoma to promote melanoma cell death.

The present work was undertaken to determine if TPP-based drugs could be found that selectively kill melanoma cells (as compared to non-malignant melanocytes) without the need for a second compound that is an inhibitor of hydroperoxide metabolism. The present data obtained from a high throughput screen, identified ten TPP-based drugs that selectively kill melanoma cells, relative to non-malignant melanocytes. Furthermore, measurements of basal oxygen consumption rates in human A375 melanoma cells (186 amol/s/cell) were significantly higher (>2×) than all other human cancer cell lines tested to, suggesting that melanoma cells may demonstrate a hyperpolarized membrane potential and be susceptible to TPP based therapy. (Wagner et al., The rate of oxygen utilization by cells. Free Radic Biol Med, 2011. 51(3):700-712. PMID:21664270.) As new knowledge of melanoma biology is revealed, new therapies have emerged including alkylating agents, immunological therapies, and kinase inhibitors. The present work is innovative because no other therapy exploits differences in mitochondrial oxidative metabolism in melanoma cells vs. non-malignant cells to selectively kill melanoma cells.

TPP molecules have long been used to study mitochondrial biophysics, and are under investigation as delivery agents for mitochondrial targeted drugs. The present data demonstrate that TPP-based drugs can be further engineered to preferentially kill melanoma cells via mechanisms of increased oxidative stress and disruption of specific mitochondrial electron transport chain (ETC) proteins (FIG. 1). In addition, the present data indicate that treating melanoma tumor bearing animals with 10-TPP slowed tumor growth and was well-tolerated. These findings show the possibility of cancer cell killing mediated by TPP-based compounds to improve the efficacy of chemotherapy for metastatic melanoma.

Triphenylphosphonium Salts

Triphenylphosphonium (TPP) salts can be reacted with alcohols, alkyl halides, and carboxylic acids, which allow them to be used as starting materials for the synthesis of a large variety of chemical derivatives, e.g., therapeutic agents. Charged molecules generally cannot pass through cell membranes without the assistance of transporter proteins because of the large activation energies need to remove of associated water molecules. In the TPP molecules, however, the charge is distributed across the large lipophilic portion of the phosphonium ion, which significantly lowers this energy requirement, and allows the TPP to pass through lipid membranes. The phosphonium salts accumulate in mitochondria due to the relatively highly negative potential inside the mitochondrial matrix. The compositions of the present invention utilize therapeutic agents that have activity in treating cancer cells, in that the therapeutic agents preferentially localize to cancer cells, as compared to the comparable normal cells because cancer cells are often characterized by abnormal mitochondrial oxidative metabolism (Aykin-Burns N, Ahmad I M, Zhu Y, Oberley L W, and Spitz D R: Increased levels of superoxide and hydrogen peroxide mediate the differential susceptibility of cancer cells vs. normal cells to glucose deprivation. Biochem. J. 2009; 418:29-37. PMID: 189376440) and altered mitochondrial membrane potential (Chen L B: Mitochondrial membrane potential in living cells, Ann. Rev. Cell Biol. 1988; 4:155-81), relative to normal cells.

In certain embodiments, the therapeutic agent comprises a triphenylphosphonium (TPP) molecule or a pharmaceutically acceptable salt thereof. As used herein, the term triphenylphosphonium is any molecule containing a triphenylphosphine cation (⁺PPh₃) moiety.

In certain embodiments, the present invention provides a composition comprising a compound of formula I:

Ph₃P⁺-L-W Y⁻ I

wherein:

W is selected from:

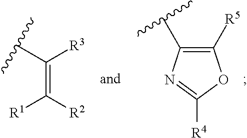

L is absent, (C₁-C₁₂)alkyl, (C₁-C₁₂)alkylene, —(CH₂CH₂O)ₙM-, —C(═O)NR^{L1}- or —NR^{L1}C(═S)NR^{L1}—;

n is 1 to 12;

M is absent or —CH₂CH₂—;

R^{L1} is H or (C₁-C₆)alkyl;

R¹ is halo or —NHC(═O)Rₐ;

R² is halo, SR_{b} or —C(═O)NHR_{c};

R³ is —NH(C═O)R_{d}, —NH(C═O)NHR_{d} or phenyl wherein any phenyl of R³ is optionally substituted with one or more halo, (C₁-C₃)alkyl, (C₁-C₃)haloalkyl or —O(C₁-C₃)alkyl;

R⁴ is (C₁-C₆)alkyl or phenyl wherein any phenyl of R⁴ is optionally substituted with one or more halo, (C₁-C₃)alkyl, (C₁-C₃)haloalkyl or —O(C₁-C₃)alkyl;

R⁵ is —S(C₁-C₆)alkyl or —N((C₁-C₆)alkyl)₂;

Rₐ is phenyl optionally substituted with one or more halo, (C₁-C₃)alkyl, (C₁-C₃)haloalkyl or —O(C₁-C₃)alkyl;

R_{b} is phenyl optionally substituted with one or more halo, (C₁-C₃)alkyl, (C₁-C₃)haloalkyl or —O(C₁-C₃)alkyl;

R_{c} is phenyl optionally substituted with one or more halo, (C₁-C₃)alkyl, (C₁-C₃)haloalkyl or —O(C₁-C₃)alkyl;

R_{d} is independently phenyl optionally substituted with one or more halo, (C₁-C₃)alkyl, (C₁-C₃)haloalkyl or —O(C₁-C₃)alkyl; and

Y is a counterion;

or a pharmaceutically acceptable salt thereof, and a pharmaceutically acceptable diluent or carrier.

In certain embodiments, L is absent or is (C₁-C₁₂)alkyl. In certain embodiments, W is

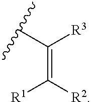

In certain embodiments, R¹ is halo or —NHC(═O)phenyl. In certain embodiments, R¹ is chloro. In certain embodiments, R² is halo, SPhCl or —C(═O)NHPh. In certain embodiments, R² is cloro. In certain embodiments, R³ is phenyl. In certain embodiments, R³ is —NH(C═O)R_{d} wherein R_{d} is phenyl substituted with fluoro or methyl, or is —NH(C═O)NHR_{d}, wherein R_{d} is phenyl substituted with cloro and (C₁)haloalkyl.

In certain embodiments, W is

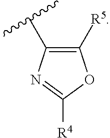

In certain embodiments, R₄ is (C₁-C₆)alkyl or phenyl wherein any phenyl of R⁴ is optionally substituted with one or more halo. In certain embodiments, R⁴ is phenyl. In certain embodiments, R⁴ is phenyl substituted with one or more cloro. In certain embodiments, R⁴ is (C₁-C₆)alkyl. In certain embodiments, R⁵ is —S(C₁-C₆)alkyl or —N((C₁-C₆)alkyl)₂. In certain embodiments, R⁵ is —S(C₁-C₄)alkyl or —N(CH₃)₂.

In certain embodiments, the compound is selected from

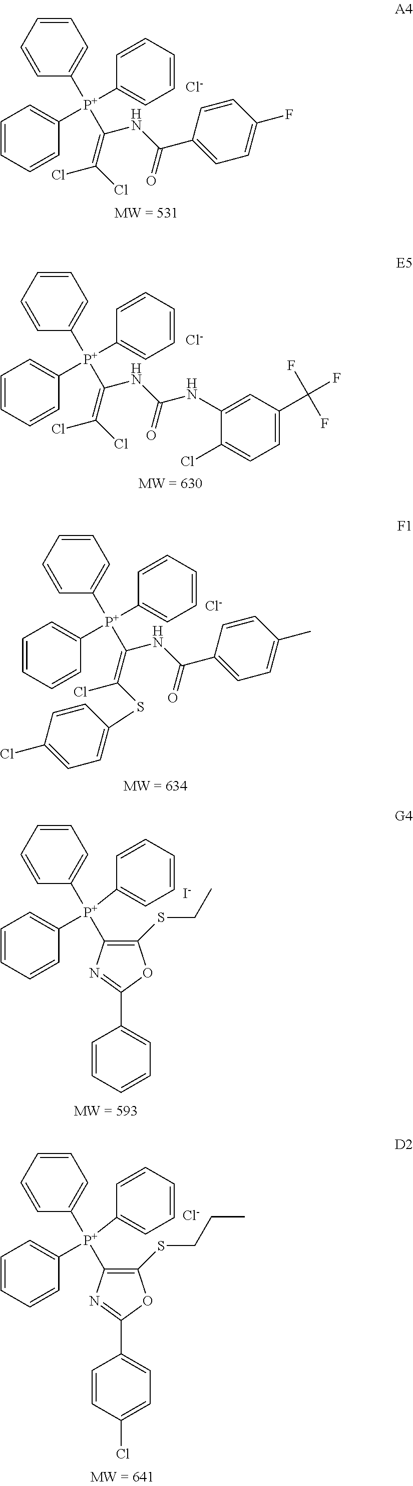

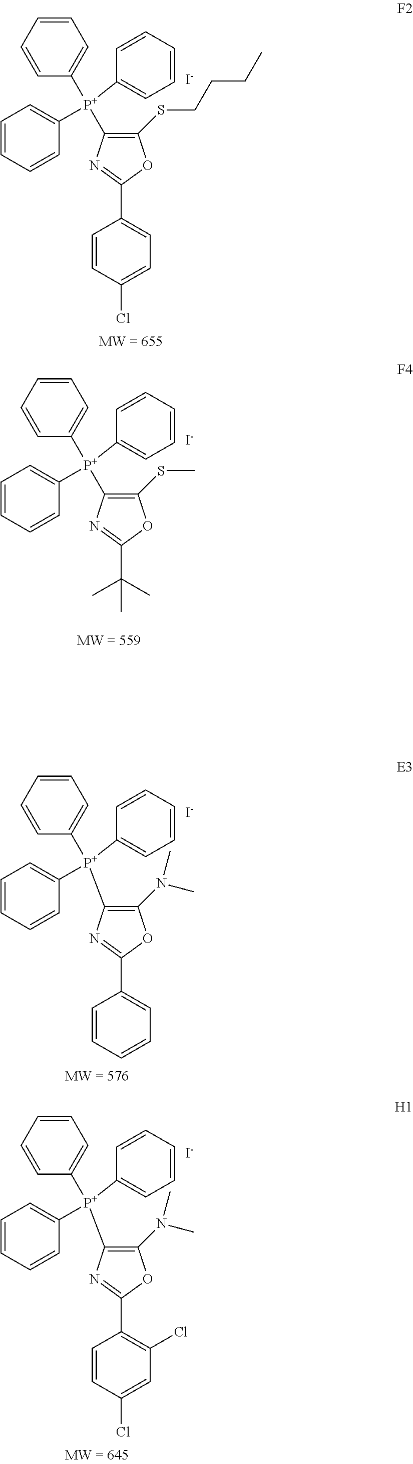

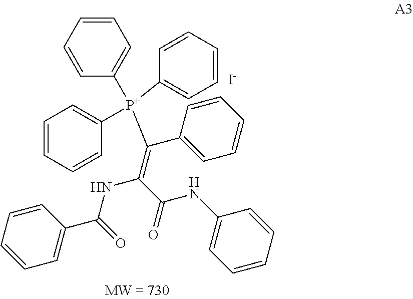 [SMILES: O=C(Nc1ccccc1)/C(NC(=O)c1ccccc1)=C(\c1ccccc1)[P+](c1ccccc1)(c1ccccc1)c1ccccc1 ; [I-]]

Inhibitors of Hydroperoxide Metabolism

In certain embodiments, inhibitors of hydroperoxide metabolism via glutathione and/or thioredoxin dependent pathways are added to a composition including a therapeutic agent that selectively enhances clonogenic cell killing via oxidative stress and accumulation of oxidative damage to critical biomolecules (i.e., proteins, lipids, and nucleic acids), in human cancer cells, relative to normal human cells. This selective property of the drug combination(s) for clonogenically inactivating cancer cells is the result of inherent differences in pro-oxidant levels generated in cancer vs. normal cells as by products of oxidative and reductive metabolism necessary for maintenance of cell viability and reproduction. More specifically, cancer cells (relative to normal cells) demonstrate increased levels of reactive oxygen species (i.e., superoxide, hydroperoxides, and reactive species derived from the oxidation of proteins, lipids, and nucleic acids) due to fundamental differences in cancer vs. normal cell metabolism of oxygen. In certain embodiments, the addition of these inhibitors of hydroperoxide metabolism to a composition including TTP enhances the efficacy of conventional radiation and chemotherapies used to treat human cancers.

In certain embodiments, the inhibitors of hydroperoxide metabolism are L-buthionine-[S,R]-sulfoximine (BSO), (S-triethylphosphinegold(I)-2,3,4,6-tetra-O-acetyl-1-thio-b-Dglucopyranoside Auranofin (AUR), or a combination of BSO and AUR. BSO and AUR or a combination of these two compounds are employed to inhibit thiol mediated hydroperoxide metabolism by both glutathione- and thioredoxin-dependent pathways which causes oxidative stress and accumulation of oxidative damage to critical biomolecules (i.e., proteins, lipids, and nucleic acids) in cancer versus normal cells resulting in cancer cell specific clonogenic cell killing in both early progenitor cancer stem cells as well as all other cancer cells capable of continued mitotic activity. In certain embodiments, other compounds are used for this purpose, including inhibitors of catalase (i.e., 3-aminotriazole), inhibitors of glucose metabolism (i.e., bromopyruvate and 2-deoxyglucose), inhibitors of peroxiredoxins, inhibitors of glutathione peroxidases, inhibitors of dehydrogenase enzymes that regenerate NADPH, inhibitors of thioredoxin reductase, inhibitors of glutathione reductase, inhibitors of glutathione transferases, and inhibitors of transcription factors as well as signal transduction proteins that regulate thiol mediated hydroperoxide metabolism (i.e., Nrf-2, AP-1, NFkB, AKT, ERK1/2, p38, EGFR, and IGFR). In certain embodiments, enhancement of the efficacy of a composition including TTP with inhibitors of hydroperoxide metabolism include feeding patients diets high in respiratory directed substrates including ketogenic diets, Atkins style diets, and pharmacological doses of IV vitamin C which would be expected to further enhance the differential production of pro-oxidants mentioned previously in cancer vs. normal tissues.

Compositions to Kill Cancer Cells Via Oxidative Stress

The present invention provides compositions to kill cancer cells via mitochondrial targeted intervention that leads to cancer cell specific cell death by mechanisms including increased oxidative stress. In certain embodiments therapeutic agents are used to selectively kill cancer cells. In certain embodiments combinations of therapeutic agents and inhibitors of hydroperoxide metabolism are combined into a single composition. In other embodiments, the two components are administered individually or sequentially. In some embodiments of the invention, the effective amount of the therapeutic agent and the inhibitors of hydroperoxide metabolism (e.g., that is administered to the subject) does not significantly affect the viability of comparable normal cells. For example, the effective amount causes the killing of less than 100% (e.g., less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5%) of the comparable normal cells. For example, the composition could kill breast cancer cells present in a mammal, but kill fewer than 100% of the normal breast cells, e.g., only 5% of the normal breast cells.

Methods of Treatment

The terms “treat” and “treatment” refer to both therapeutic treatment and prophylactic or preventative measures, wherein the object is to prevent or decrease an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The therapeutic agent and inhibitors of hydroperoxide metabolism may be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The dosage of the therapeutic agent and inhibitors of hydroperoxide metabolism will vary depending on age, weight, and condition of the subject. Treatment may be initiated with small dosages containing less than optimal doses, and increased until a desired, or even an optimal effect under the circumstances, is reached. In general, the dosage is about 1 μg/kg up to about 100 μg/kg body weight, e.g., about 2 μg/kg to about μg/kg body weight of the subject, e.g., about 8 μg/kg to about 35 μg/kg body weight of the subject. Higher or lower doses, however, are also contemplated and are, therefore, within the confines of this invention. A medical practitioner may prescribe a small dose and observe the effect on the subject's symptoms. Thereafter, he/she may increase the dose if suitable. In general, the therapeutic agent and inhibitors of hydroperoxide metabolism are administered at a concentration that will afford effective results without causing any unduly harmful or deleterious side effects, and may be administered either as a single unit dose, or if desired in convenient subunits administered at suitable times.

A pharmaceutical composition of the invention is formulated to be compatible with its intended route of administration. For example, the therapeutic agent may be introduced directly into the cancer of interest via direct injection. Additionally, examples of routes of administration include oral, parenteral, e.g., intravenous, slow infusion, intradermal, subcutaneous, oral (e.g., ingestion or inhalation), transdermal (topical), transmucosal, and rectal administration. Such compositions typically comprise the therapeutic agent and inhibitors of hydroperoxide metabolism and a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art.

Solutions or suspensions can include the following components: a sterile diluent such as water for injection, saline solution (e.g., phosphate buffered saline (PBS)), fixed oils, a polyol (for example, glycerol, propylene glycol, and liquid polyetheylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol or sorbitol, and sodium chloride in the composition. Prolonged administration of the injectable compositions can be brought about by including an agent that delays absorption. Such agents include, for example, aluminum monostearate and gelatin. The parenteral preparation can be enclosed in ampules, disposable syringes, or multiple dose vials made of glass or plastic.

It may be advantageous to formulate compositions in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms of the invention are dependent upon the amount of a compound necessary to produce the desired effect(s). The amount of a compound necessary can be formulated in a single dose, or can be formulated in multiple dosage units. Treatment may require a one-time dose, or may require repeated doses.

The invention will now be illustrated by the following non-limiting Examples.

### Example 1

Experiments were conducted to test the hypothesis that TPP-based therapies can be developed to selectively kill melanoma cells relative to non-malignant melanocytes by exploiting differences in mitochondrial oxidative metabolism of melanoma cells relative to non-malignant melanocytes. Experiments involving animals and radioactive materials were carried out under approved protocols.

Mitochondrial-targeted TPP-based drugs that selectively kill melanoma cells relative to non-malignant immortalized melanocytes have been identified by a high throughput screen. To test the working hypothesis, MTT (3-(4-5-Dimethylthiazol-2-yl)-2-5-diphenyltetrazolium bromide) cell viability assays were used to screen 48 TPP compounds for selective melanoma cell killing relative to non-malignant melanocytes (Table 1).

TABLE 1
TPP-based compounds tested for selectively killing melanoma cells relative to
non-malignant melanocytes

| Compound | |
|---|---|
| No. | Name of Compound |
| 1 | [5-(allylthio)-2-phenyl-1,3-thiazol-4-yl](triphenyl)phosphonium iodide |
| 2 | {2,2-dichloro-1-[({[2-chloro-5- |
| | (trifluoromethyl)phenyl]amino}carbonyl)amino]vinyl}(triphenyl)phosphonium chloride |
| 3 | {2-chloro-2-[(4-chlorophenyl)thio]-1-[(4- |
| | methylbenzoyl)amino]vinyl}(triphenyl)phosphonium chloride |
| 4 | [2-(2,4-dichlorophenyl)-5-(dimethylamino)-1,3-oxazol-4-yl](triphenyl)phosphonium iodide |
| 5 | hexadecyltriphenylphosphonium |
| 6 | [5-(allylthio)-2-phenyl-1,3-oxazol-4-yl](triphenyl)phosphonium iodide |
| 7 | [5-(allylthio)-2-phenyl-1,3-thiazol-4-yl](triphenyl)phosphonium iodide |
| 8 | [5-(allylthio)-2-(4-methylphenyl)-1,3-oxazol-4-yl](triphenyl)phosphonium iodide |
| 9 | [2-(4-chlorophenyl)-5-(ethylthio)-1,3-oxazol-4-yl](triphenyl)phosphonium iodide |
| 10 | [5-(butylthio)-2-(4-chlorophenyl)-1,3-oxazol-4-yl](triphenyl)phosphonium iodide |
| 11 | [2-(4-chlorophenyl)-5-(propylthio)-1,3-oxazol-4-yl](triphenyl)phosphonium iodide |
| 12 | [5-(ethylthio)-2-phenyl-1,3-oxazol-4-yl](triphenyl)phosphonium iodide |
| 13 | decyltriphenylphosphonium |
| 14 | [5-(dimethylamino)-2-phenyl-1,3-oxazol-4-yl](triphenyl)phosphonium iodide |
| 15 | [2-(2-chlorophenyl)-5-(methylthio)-1,3-oxazol-4-yl](triphenyl)phosphonium iodide |
| 16 | [2-tert-butyl-5-(methylthio)-1,3-oxazol-4-yl](triphenyl)phosphonium iodide |
| 17 | {2-(butylthio)-1-[(4-methylbenzoyl)amino]vinyl}(triphenyl)phosphonium iodide |
| 18 | [5-(ethylthio)-2-(2-phenylvinyl)-1,3-oxazol-4-yl](triphenyl)phosphonium iodide |
| 19 | 3-methoxy-1-[(4-nitrobenzoyl)amino]-3-oxo-2-(triphenylphosphonio)-1-propene-1-thiolate |
| 20 | (5-{[1-(ethoxycarbonyl)propyl]thio}-2-phenyl-1H-imidazol-4-yl)(triphenyl)phosphonium |
| | bromide |
| 21 | [3-anilino-2-(benzoylamino)-3-oxo-1-phenyl-1-propen-1-yl](triphenyl)phosphonium iodide |
| 22 | (1-benzoyl-2-hydroxy-1-propen-1-yl)(triphenyl)phosphonium chloride |
| 23 | (1-{[(tert-butylamino)carbonyl]amino}-2,2-dichlorovinyl)(triphenyl)phosphonium chloride |
| 24 | [5-(4-morpholinyl)-1,3-oxazol-4-yl](triphenyl)phosphonium iodide |
| 25 | (2-cyano-2-{[(4-methylphenyl)sulfonyl]amino}vinyl)(triphenyl)phosphonium chloride |
| 26 | [5-(ethylthio)-2-methyl-1,3-oxazol-4-yl](triphenyl)phosphonium iodide |
| 27 | pentyltriphenylphosphonium |
| 28 | {2-[(4-chlorophenyl)thio]-1-[(2,2-dimethylpropanoyl)amino]vinyl}(triphenyl)phosphonium |
| | iodide |
| 29 | [1-(ethoxycarbonyl)-2-hydroxy-1-propen-1-yl](triphenyl)phosphonium bromide |
| 30 | [1-(acetylamino)-2-chloro-2-(methylthio)vinyl](triphenyl)phosphonium chloride |
| 31 | N-[2-(4-fluorophenyl)-1-(triphenylphosphonio)vinyl]benzenecarboximidate |
| 32 | 2-(benzoylamino)-3-methoxy-3-oxo-1-(triphenylphosphonio)-1-propene-1-thiolate |
| 33 | [2-(4-methylphenyl)-5-(propylthio)-1,3-oxazol-4-yl](triphenyl)phosphonium iodide |
| 34 | 2-(acetylamino)-1-[(4-chlorophenyl)thio]-2-(triphenylphosphonio)ethylenethiolate |
| 35 | {1-[(4-chlorophenyl)diazenyl]-2,3-dioxo-3-phenylpropyl}(triphenyl)phosphonium |
| 36 | {2,2-dichloro-1-[(2,2-dimethylpropanoyl)amino]vinyl}(triphenyl)phosphonium chloride |
| 37 | [4-mercapto-2-(4-methylphenyl)-6-oxo-1,6-dihydro-5-pyrimidinyl](triphenyl)phosphonium |
| | chloride |
| 38 | [1-(benzoylamino)-2-(ethylthio)vinyl](triphenyl)phosphonium iodide |
| 39 | [1-(benzoylamino)-2,2-bis(ethylthio)vinyl](triphenyl)phosphonium chloride |
| 40 | (4-oxo-2-(trichloromethyl)-3,4-dihydro-2H-1,3,5-thiadiazin-6-yl)(triphenyl-lambda~5~- |
| | phosphanyl)acetonitrile |
| 41 | {1-[(4-methylbenzoyl)amino]-2-[(2-oxo-2-phenylethyl)thio]vinyl}(triphenyl)phosphonium |
| | bromide |
| 42 | 4-(triphenylphosphoranyl)benzoic acid |
| 43 | {4-[(2-methoxy-2-oxoethyl)thio]-2-methyl-6-oxo-1,6-dihydro-5- |
| | pyrimidinyl}(triphenyl)phosphonium chloride |
| 44 | N-(2-nitrophenyl)-N′-[(triphenylphosphonio)methyl]urea |
| 45 | [1-(benzoylamino)-2-(propylthio)vinyl](triphenyl)phosphonium iodide |
| 46 | 2-(benzoylamino)-3-(triphenylphosphonio)acrylate |
| 47 | [1-[(2,2-dimethylpropanoyl)amino]-2-(methylthio)vinyl](triphenyl)phosphonium iodide |
| 48 | [2-(benzoylamino)-3-methoxy-3-oxo-1-phenyl-1-propen-1-yl](triphenyl)phosphonium |
| | iodide |

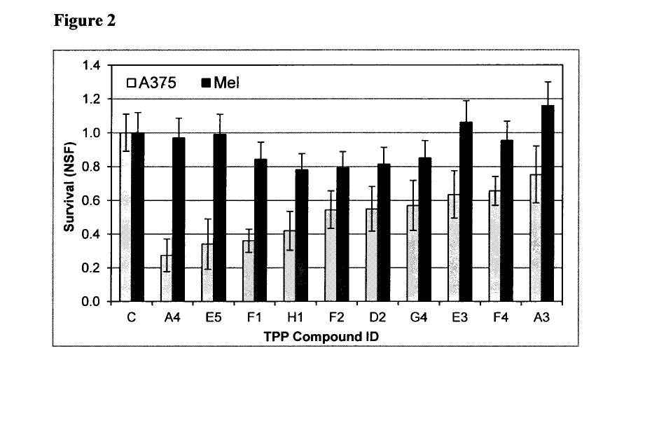

This library was selected based on database searches of commercially-available TPP compounds, which include the TPP moiety and differ in the composition of the molecular side chain (e.g., FIGS. 1, 2).

Target cells were BRAF mutant A375 human melanoma. This is important because the BRAF mutation is the most common genetic mutation in human metastatic melanoma (50-70%). (Gray-Schopfer eta 1., Melanoma biology and new targeted therapy. Nature, 2007. 445(7130):851-857. PMID:17314971; Davies et al., Mutations of the BRAF gene in human cancer. Nature, 2002. 417(6892):949-954. PMID:12068308; Gray-Schopfer V C, Karasarides M, Hayward R, and Marais R, Tumor necrosis factor-alpha blocks apoptosis in melanoma cells when BRAF signaling is inhibited. Cancer Res, 2007. 67(1):122-129. PMID:17210691; Tsao et al., Genetic interaction between NRAS and BRAF mutations and PTEN/MMAC1 inactivation in melanoma. J Invest Dermatol, 2004. 122(2):337-341. PMID:15009714.) All 48 TPP compounds were screened against A375 cells in duplicate (n=2) and non-tumorigenic immortalized melanocytes (n=1) from ATCC (#CRL-2770). Cells were incubated with test compounds at 2 μM for 24 h. Following incubation, medium was aspirated, MTT solution in fresh medium was added and incubated for 1 h. Cells were washed, and DMSO was added to dissolve the colorimetric dye taken up by viable cells and analyzed according to established protocols.

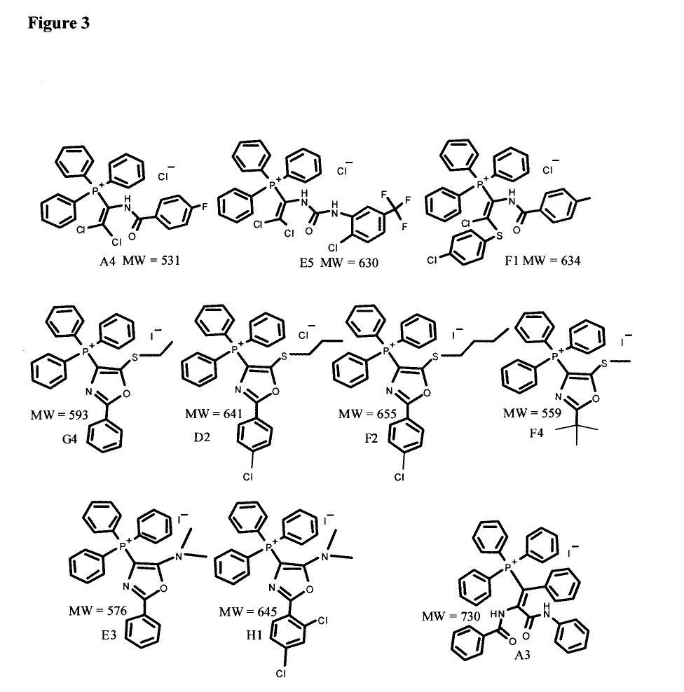

Ten compounds that selectively killed human melanoma A375 cells while being relatively non-toxic to non-tumorigenic melanocytes were identified (FIG. 3, Table 2).

TABLE 2

| ID No. | Compound |
|---|---|
| A4 | [5-(allylthio)-2-phenyl-1,3-thiazol-4- |
| | yl](triphenyl)phosphonium iodide |
| E5 | {2,2-dichloro-1-[({[2-chloro-5- |
| | (trifluoromethyl)phenyl]amino}carbonyl)amino]vinyl} |
| | (triphenyl)phosphonium chloride |
| F1 | {2-chloro-2-[(4-chlorophenyl)thio]-1-[(4- |
| | methylbenzoyl)amino]vinyl}(triphenyl)phosphonium chloride |
| G4 | [5-(ethylthio)-2-phenyl-1,3-oxazol-4- |
| | yl](triphenyl)phosphonium iodide |
| D2 | [2-(4-chlorophenyl)-5-(propylthio)-1,3-oxazol-4- |
| | yl](triphenyl)phosphonium iodide |
| F2 | [5-(butylthio)-2-(4-chlorophenyl)-1,3-oxazol-4- |
| | yl](triphenyl)phosphonium iodide |
| F4 | [2-tert-butyl-5-(methylthio)-1,3-oxazol-4- |
| | yl](triphenyl)phosphonium iodide |
| E3 | [5-(dimethylamino)-2-phenyl-1,3-oxazol-4- |
| | yl](triphenyl)phosphonium iodide |
| H1 | [2-(2,4-dichlorophenyl)-5-(dimethylamino)-1,3-oxazol-4- |
| | yl](triphenyl)phosphonium iodide |
| A3 | [3-anilino-2-(benzoylamino)-3-oxo-1-phenyl-1-propen-1- |
| | yl](triphenyl)phosphonium iodide |

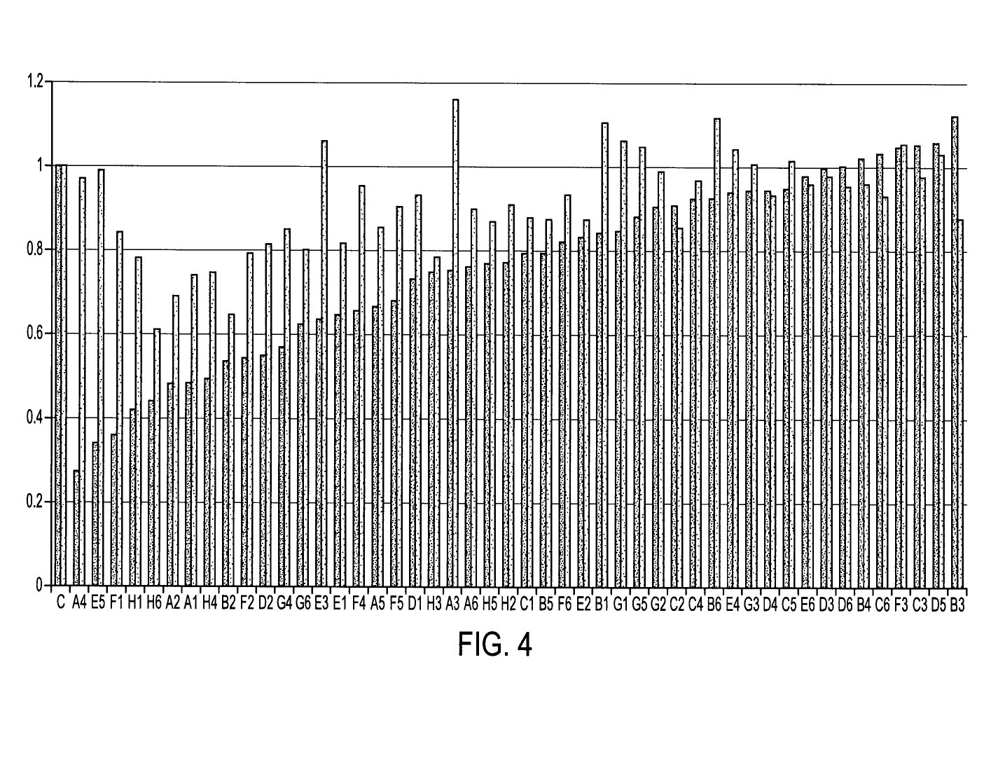

These results identified specific side-chain structural similarities that impart melanoma specific cytotoxicity under the conditions of this experiment. For example, TPP based compounds that included vinyl chloride substituents and amide-bond and amide-like linkages to terminal benzyl groups provided the highest differential melanoma specific cytotoxicity (FIG. 3; A1-A3). A second group of TPP-based compounds that promoted melanoma specific cell death relative to non-malignant melanocytes included oxazole linkages to terminal benzyl groups, thioether groups, tertiary amine functional groups, (FIG. 3; B1-3). Additional compounds were also identified (FIG. 4) that promoted melanoma specific cell death relative to non-malignant melanocytes. All compounds identified as selectively toxic to melanoma cells relative to non-malignant melanocytes included a terminal benzyl group that in some cases included halo substituents or other substituents. These results support the hypothesis that specific structural features of these TPP based drugs lead to selective melanoma cell killing.

Materials and Methods:

A375 human melanoma (catalog no. CRL1619™) cells were purchased from ATCC®. All cells were cultured in 150 cm² culture flasks at 37° C. in a humidified 5% CO₂ incubator and detached with 0.25% trypsin-EDTA (GIBCO®, catalog no. 25200). A375 cells were cultured in high glucose DMEM medium (GIBCO®, catalog no. 11965) supplemented with 1% PenStrep (GIBCO®, catalog no. 15140) and 10% FBS (GIBCO®, catalog no. 26140). MeWo cells were cultured in 1:1 DMEM:F12 (VENDOR INFO) medium supplemented with 1% PenStrep and 10% FBS. Melanocytes were cultured in Ham's F10 medium (GIBCO®, catalog no 11550) supplemented with 7% horse serum (Hyclone®, catalog no. SH30074.03) and 50 ng/mL 12-myristate 13-acetate (TPA) (Sigma®, catalog no. P-8139). Experiments were performed with cells at or below passage twenty.

The MTT (3-(4-5-Dimethylthiazol-2-yl)-2-5-diphenyltetrazolium bromide) method was used in a screening assay to screen a library of commercially available TPP-based compounds to test the hypothesis that specific structural features of TPP-based drugs could be identified that promote cancer cell specific cytotoxicity relative to non-malignant cells. For these assays, A375 (2.5×10⁴ cells per well) and melanocytes (5.0×10⁴ cells per well) were plated and incubated for 24 h. Following incubation, media was aspirated and compounds were added at a final concentration of 2 μM in 200 μL media using a Hamilton Star liquid handler. After 24 h, media was aspirated and 200 μL MTT solution (5 mg/mL MTT in PBS) was added and incubated for 1.5 h. MTT solution was aspirated and 150 μL DMSO was added to dissolve the colorimetric dye taken up by viable cells. Absorbance was measured using a Perkin Elmer Envision Multimode high-speed plate reader at 590 nm. Ten compounds that selectively killed human melanoma A375 cells while being relatively non-toxic to non-tumorigenic melanocytes were identified by this method.

### Example 2

MTT viability analysis of melanoma cells versus non-malignant melanocyte cells looking at the effect of variation in TPP molecular chain length and treatment time. A375 human melanoma cells in (A), MeWo human melanoma cells in (B), and non-malignant melanocyte cells in (C) were treated with TPP derivatives at a 0.5 μM concentration for 24 h, 48 h, and 72 h. N=4 for each bar. P<0.001. Results show no significant decrease in viability in A375 and MeWo melanoma cells treated with pentyl-TPP, decyl-TPP, and hexadecyl-TPP variants for 24 h. Decyl-TPP led to ˜>30% decrease in A375 and MeWo cell viability at 48 h and 72 h, while hexadecyl-TPP resulted in ˜>50% decrease in A375 and MeWo cell viability and 48 h and 72 h. No significant decrease in non-malignant melanocyte cell viability was observed under these conditions for the pentyl-TPP, decyl-TPP, and hexadecyl-TPP variants at 24 h and 48 h. The hexadecyl-TPP variant led to a ˜25% decrease in melanocyte viability at 72 h, and these effects were not observed in cells treated with pentyl-TPP and decyl-TPP for 72 h. These data demonstrate that there is a time dependence and structure-activity relationship between side chain length and decreased viability in melanoma cells, while inclusion of longer length side chains and treatment time has little effect on non-malignant melanocyte cell viability to TPP.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A pharmaceutical composition comprising a compound of formula I:
Ph₃P⁺-L-W Y⁻ I
wherein:
W is selected from: 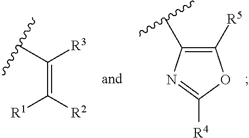
L is absent, (C₁-C₁₂)alkyl, (C₁-C₁₂)alkylene, —(CH₂CH₂O)ₙM-, —C(═O)NR^{L1}- or —NR^{L1}C(═S)NR^{L1}—;
n is 1 to 12;
M is absent or —CH₂CH₂—;
R^{L1} is H or (C₁-C₆)alkyl;
R¹ is halo or —NHC(═O)Rₐ;
R² is halo, SR_{b} or —C(═O)NHR_{c};
R³ is —NH(C═O)R_{d}, —NH(C═O)NHR_{d} or phenyl wherein any phenyl of R³ is optionally substituted with one or more halo, (C₁-C₃)alkyl, (C₁-C₃)haloalkyl or —O(C₁-C₃)alkyl;
R⁴ is (C₁-C₆)alkyl or phenyl wherein any phenyl of R⁴ is optionally substituted with one or more halo, (C₁-C₃)alkyl, (C₁-C₃)haloalkyl or —O(C₁-C₃)alkyl;
R⁵ is —S(C₁-C₆)alkyl or —N((C₁-C₆)alkyl)₂;
Rₐ is phenyl optionally substituted with one or more halo, (C₁-C₃)alkyl, (C₁-C₃)haloalkyl or —O(C₁-C₃)alkyl;
R_{b} is phenyl optionally substituted with one or more halo, (C₁-C₃)alkyl, (C₁-C₃)haloalkyl or —O(C₁-C₃)alkyl;
R_{c} is phenyl optionally substituted with one or more halo, (C₁-C₃)alkyl, (C₁-C₃)haloalkyl or —O(C₁-C₃)alkyl;
R_{d} is independently phenyl optionally substituted with one or more halo, (C₁-C₃)alkyl, (C₁-C₃)haloalkyl or —O(C₁-C₃)alkyl; and
Y is a counterion;
or a pharmaceutically acceptable salt thereof, and a pharmaceutically acceptable diluent or carrier.

2. The composition of claim 1, wherein L is absent or (C₁-C₁₂)alkyl.

3. The composition of claim 1, wherein W is

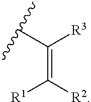

4. The composition of claim 3, wherein R¹ is halo or —NHC(═O)phenyl.

5. The composition of claim 3, wherein R¹ is chloro.

6. The composition of claim 3, wherein R¹ is —NHC(═O)phenyl.

7. The composition of claim 3, wherein R² is halo, SPhC1 or —C(═O)NHPh.

8. The composition of claim 3, wherein R² is cloro.

9. The composition of claim 3, wherein R³ is phenyl.

10. The composition of claim 3, wherein R³ is —NH(C═O)R_{d} wherein R_{d} is phenyl substituted with fluoro or methyl, or is —NH(C═O)NHR_{d}, wherein R_{d} is phenyl substituted with cloro and (C₁)haloalkyl.

11. The composition of claim 1 or 2, wherein W is

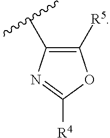

12. The composition of claim 11, wherein R⁴ is (C₁-C₆)alkyl or phenyl wherein any phenyl of R⁴ is optionally substituted with one or more halo.

13. The composition of claim 11, wherein R⁴ is phenyl.

13. The composition of claim 11, wherein R⁴ is phenyl substituted with one or more cloro.

14. The composition of claim 11, wherein R⁴ is (C₁-C₆)alkyl.

15. The composition of claim 11, wherein R⁵ is —S(C₁-C₆)alkyl or —N((C₁-C₆)alkyl)₂.

16. The composition of claim 11, wherein R⁵ is —S(C₁-C₄)alkyl or —N(CH₃)₂.

17. The composition of claim 1, wherein the compound is selected from

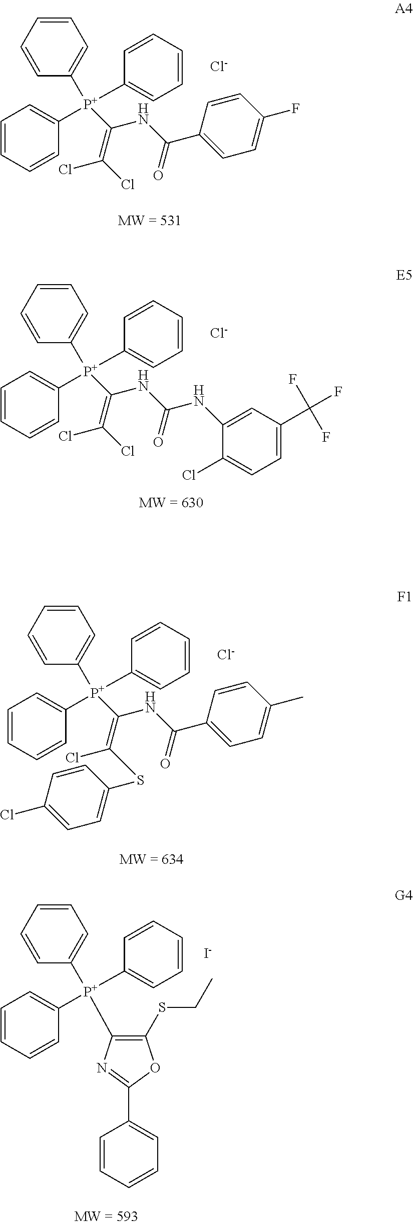

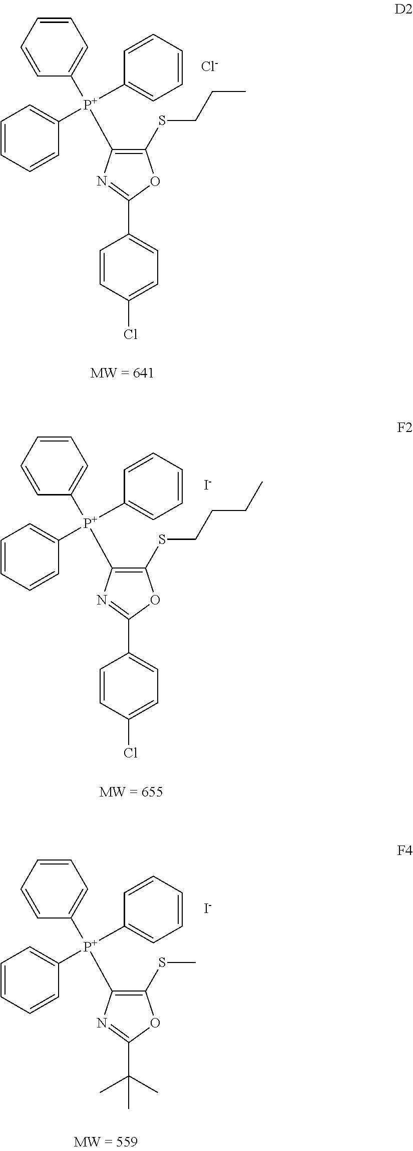 [SMILES: CCCSc1oc(-c2ccc(Cl)cc2)nc1[P+](c1ccccc1)(c1ccccc1)c1ccccc1 ; [Cl-] ; CCCCSc1oc(-c2ccc(Cl)cc2)nc1[P+](c1ccccc1)(c1ccccc1)c1ccccc1 ; [I-] ; CSc1oc(C(C)(C)C)nc1[P+](c1ccccc1)(c1ccccc1)c1ccccc1 ; [I-]]

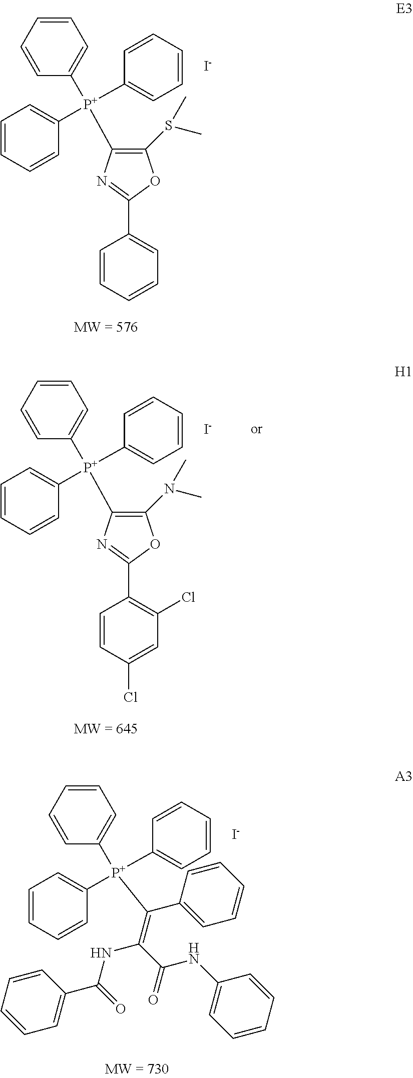

18. The pharmaceutical composition of claim 17, further comprising an inhibitor of glutathione synthesis or hydroperoxide metabolism.

19. The pharmaceutical composition of claim 18, wherein the inhibitor of hydroperoxide metabolism comprises L-buthionine-[S,R]-sulfoximine (BSO), or (S-triethylphosphinegold(I)-2,3,4,6-tetra-O-acetyl-1-thio-b-Dglucopyranoside Auranofin (AUR), or a combination of BSO and AUR.

20. The pharmaceutical composition of claim 1, wherein the inhibitor of hydroperoxide metabolism comprises catalase, inhibitors of glucose metabolism, inhibitors of peroxiredoxins, inhibitors of glutathione peroxidases, inhibitors of dehydrogenase enzymes that regenerate NADPH, inhibitors of thioredoxin reductase, inhibitors of glutathione reductase, inhibitors of glutathione transferases, inhibitors of transcription factors, or inhibitors of a signal transduction protein that regulate thiol mediated hydroperoxide metabolism.

21-23. (canceled)

24. A method for treating cancer in a mammal, comprising administering the composition of claim 1 to the mammal.

25. A method for inducing cellular apoptosis or clonogenic cell killing of a cancerous cell, comprising contacting the cancerous cell with an effective toxicity-inducing amount of the composition of claim 1.

26. A method for increasing the anticancer effects of a cancer therapy on a cancerous cell in a mammal, comprising contacting the cancerous cell with an effective amount of the composition of claim 1 and contacting prior to administering an additional cancer therapy.

27-28. (canceled)

29. A method for inducing oxidative stress in a cancer cell in a mammal in need of such treatment comprising administering to the mammal an effective amount of the composition of claim 1.

30-40. (canceled)

41. A method for treating cancer in a subject, comprising administering to the subject an effective amount of a composition of claim 1 and an inhibitor of hydroperoxide metabolism so as to treat the cancer.

42-43. (canceled)

44. The method of claim 24, further comprising administering pharmacological doses of IV vitamin C.

45-47. (canceled)
